(19) United States
(12) Patent Application Publication
(10) Pub. No.: US 2020/0168306 A1
(43) Pub. Date: May 28, 2020

(54) METHOD AND SYSTEM FOR SHARING ELECTRONIC MEDICAL AND HEALTH RECORDS

(71) Applicant: TAIPEI MEDICAL UNIVERSITY, Taipei City (TW)

(72) Inventors: Ray-Jade Chen, New Taipei City (TW); Yu-Sheng Lo, New Taipei City (TW); Lan-Ying Kang, Kaohsiung City (TW); Hsing-Nan Hou, New Taipei City (TW); Wan-Lin Chou, Kaohsiung City (TW)

(21) Appl. No.: 16/699,004

(22) Filed: Nov. 28, 2019

(30) Foreign Application Priority Data
Nov. 28, 2018 (TW) 107142489

(51) Int. Cl. G16H 10/60; H04L 29/06; G06K 7/14

(52) U.S. Cl. CPC G16H 10/60; G06K 7/1417; H04L 63/10

(57) ABSTRACT

Provided herein is to a method and a system for sharing electronic medical and health records. The method is operated in a serving system with a database. The system provides a software program executed by a terminal device. In the method, the serving system receives a connection request from the terminal device and verifies a user blockchain ID. The system provides one or more medical-health record indexes after the verification. When receiving an instruction for sharing medical-health record from the terminal device, the system generates a trust anchor for the terminal device. The trust anchor is provided for the terminal device to share with a shared recipient. When receiving an authorization message from the shared recipient, the system transmits the medical-health records to be shared to the shared recipient after verifying the blockchain ID and the authorization message.

### CROSS-REFERENCE TO RELATED PATENT APPLICATION

This application claims the benefit of priority to Taiwan Patent Application No. 107142489, filed on Nov. 28, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The disclosure is related a method and a system for sharing EMR/EHR records, and in particular to a method for sharing EMR/EHR record indexes with a user via blockchain, and a system thereof.

### BACKGROUND OF THE DISCLOSURE

Traditional medical institutions generally store patient data by means of paper with manually created indexes. If there is a need to access the patient data, the patient data can be found according to the indexes. The patient data can be used as a reference for follow-up diagnosis and treatment.

Such conventional paper records can also be digitalized to electronic medical records. The electronic medical records are beneficial to circulation and portability. The electronic medical records allow a doctor to deal with a patient condition more comprehensively and correctly since it is convenient for the doctor to refer to the patient records in other medical departments.

It is common practice for the traditional medical institutions to store their own medical records but with no way to share them. Through national health insurance, the medical records are often stored in specific server systems of government agencies for centralized management. However, the traditional framework of the server systems may harbor security concerns such as data leakage or malicious tampering, and may not allow the public or the patients to perform secure queries due to any flaws in the security mechanism.

The conventional authorization management for the electronic medical records stored in a database is generally based on the security mechanism such as user account, password, dedicated line and virtual private network (VPN), etc. However, it is not convenient and has no sufficient protection for the conventional way to share the medical records with others through the medical institution system and the conventional security mechanism.

### SUMMARY OF THE DISCLOSURE

The disclosure is related to a blockchain-based method for sharing EMR/EHR records and a system thereof. One of the objectives of the method is to share the EMR/EHR record indexes recorded in a blockchain network that is constituted by the blockchain technology with the features such as security, accuracy and decentralization. The blockchain-based method achieves a portable and convenient way to share the Electronic Medical Records (EMR) and Electronic Health Records (HER) with others.

According to one of the embodiments of the disclosure, the method for sharing EMR/EHR records is operated in a serving system. In the method, a serving system is provided to receive a connection request from a terminal device. In the serving system, a blockchain ID of a user of the terminal device is verified. One or more EMR/EHR record indexes can be shared according to the blockchain ID. The serving system then receives an instruction for sharing EMR/EHR records from the terminal device. A signal for sharing the EMR/EHR records that are selected from the one or more EMR/EHR record indexes is generated. A trust anchor is generated and transmitted to the terminal device.

At the user end, the user shares the trust anchor in the terminal device to a recipient to whom the records accompanying the trust anchor are shared, i.e. a shared person. When the shared person receives the trust anchor, an authorization message is generated and transmitted to the serving system.

Next, when the serving system receives the authorization message transmitted from the device of shared recipient, the serving system verifies a blockchain ID of the shared person and the authorization message. The EMR/EHR records to be shared are then transmitted to the shared person after verification.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram depicting a system framework for sharing electronic medical and health records using blockchain technology according to one embodiment of the disclosure.

FIG. 2 is a schematic diagram depicting a system framework for sharing electronic medical and health records using blockchain technology in another embodiment of the disclosure;

FIG. 3 shows a flow chart describing a process in a terminal device in a method for sharing electronic medical and health records in one embodiment of the disclosure;

FIG. 4 shows a flow chart describing a process in a server system in a method for sharing electronic medical and health records in one embodiment of the disclosure;

FIG. 5 is a schematic diagram depicting a circumstance where the EMR/EHR records are shared through mobile devices in one embodiment of the disclosure;

FIG. 6 shows a flow chart describing a method for sharing EMR/EHR records in one embodiment of the disclosure;

FIG. 7 shows a flow chart describing a utilization of blockchain technology to assist medical staff to create EMR/EHR records in the method for sharing EMR/EHR records according to one embodiment of the disclosure; and

FIG. 8 is a schematic diagram depicting a user device acquiring EMR/EHR records in one embodiment of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The disclosure relates to a method for sharing electronic medical and health records (hereinafter ‘EMR/HER’) and a system thereof. The method in particular utilizes blockchain technology that has established a trust relationship with other nodes to create indexes for indicating the EMR/EHR records for linking with a medical-health record database. The indexes are converted into decentralized records by the blockchain technology. The decentralized records allow an authorized user to access the electronic EMR/EHR records based on his level of authorization and share the records with others based on the trust relationship. The system utilizes the features of blockchain such as security, correctness and decentralization to create the indexes for the electronic EMR/EHR records. The EMR/EHR records can also be queried, established and shared in the database with consideration to security and correctness. The above-mentioned electronic EMR/EHR records can be categorized into electronic medical records (EMR) that are generated in hospitals, medical institutions or health examination centers, and electronic health records (EHR) that are generated by inspection devices. The records in the database can also include both EMRs and EHRs, and can also be the medical records or health records of patients.

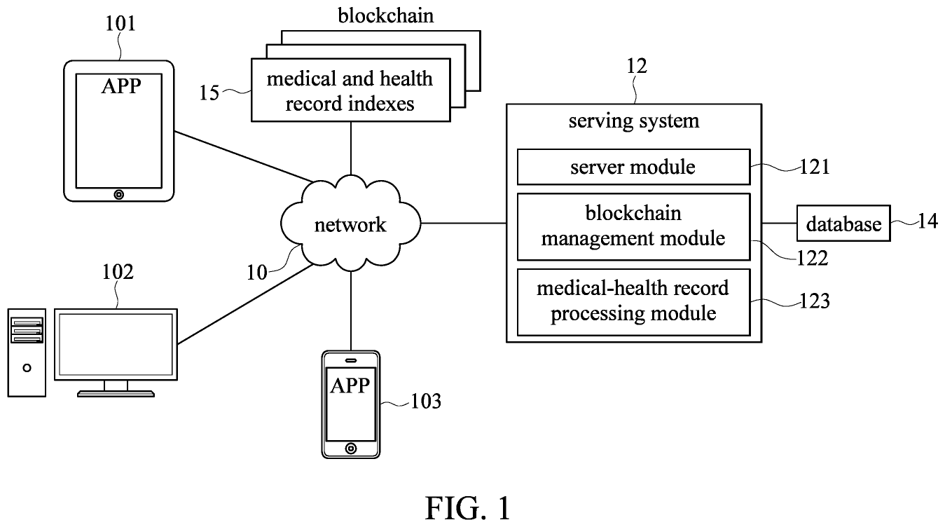

Reference is made to FIG. 1, showing a system framework that implements a system for sharing electronic EMR/EHR records according to one embodiment of the disclosure.

The main components of the system for sharing the electronic EMR/EHR records include a computer-implemented server system 12. The server system forms a cloud platform via a network 10. The cloud platform combines one or more medical-health institution systems. The server system 12 may have its own database 14 or combine one or more medical-health record databases of the one or more medical-health institution systems. The medical-health institution can be a medical institution or a health examination center. In addition to the equipment at the server end, the system also provides software programs stored in various terminal devices 101, 102 and 103. The software program can be operated in a mobile device or a computer terminal. The software program can be successfully executed after an authentication process is passed, e.g. fingerprint verification, password validation or face recognition. When the software program is executed by a processor of one of the terminal devices (101, 102, 103), the terminal device connects with the server system 12 via the network 10. A query command for querying EMR/EHR records and establishing trust relationship with others can be generated through a user interface. The query command can be adapted to retrieve the indexes for the electronic EMR/EHR records in a blockchain network 15.

The server system 12 provides functionalities that are implemented by hardware and software. The functionalities can be embodied by a server module 121, a blockchain management module 122 and a medical-health record processing module 123 in the server system 12. The server module 121 provides services such as establishing connectivity for the terminal devices 101, 102 and 103 and initiating a user interface allowing the terminal devices to connect with the server system 12 via Internet protocol, e.g. HTTP.

The blockchain management module 122 is responsible for managing blockchain IDs of users. The blockchain management module 122 allows the users to register blockchain IDs and the related certificates, and to acquire public keys, private keys and a password pertinent to the keys. The blockchain management module 122 issues the indexes for electronic EMR/EHR records and verifies the services for acquiring the indexes. For example, the blockchain management module 122 verifies the blockchain ID of a user who initiates the connection. The system therefore provides services according to the user identity. Further, the blockchain management module 122 stores the indexes for indicating the electronic EMR/EHR records by decentralized ledgers or addresses of a specific blockchain network through an encryption algorithm.

Furthermore, the blockchain management module 122 is provided in the serving system 12 to manage trust relationship among the users. The trust relationship allows the user to share his EMR/EHR records with others with blockchain IDs via the blockchain network. When the user wants to share the EMR/EHR records with the others, a temporary trust anchor is required. The temporary trust anchor is associated with a temporary authorization for a recipient to whom the records are shared, i.e. a shared person, to obtain the authorization to access the EMR/EHR records of the user.

Further, a medical-health record processing module 123 is responsible for processing the procedure to establish the database 14 for the electronic EMR/EHR records in the server system 12. The module 123 then creates the indexes for the records. In one embodiment of the disclosure, the blockchain management module 122 manages the pubic keys for the users or patients. When a new record is created by a patient, the medical-health record processing module 123 acquires the public key after receiving the record. The key allows the system to encrypt the new electronic EMR/EHR record, and to create an index for the electronic EMR/EHR record. The encrypted record is then stored into the database 14 through the module 123. The index is uploaded to the blockchain network 15 by a decentralized ledger protocol using an encryption algorithm, and the index forms one of the records in the blockchain network 15.

Through the indexes over the blockchain network 15, the server system 12 is able to access the medical-health record database of other medical-health institution systems. In addition, the server system 12 allows its users to create electronic EMR/EHR records and forms the indexes that link to the records by the blockchain technology. The main components of the electronic EMR/EHR records stored in the database 14 are patient IDs, timestamps with respect to the records, details related to the records, and queryable database addresses that are linked with the indexes.

The terminal devices 101, 102 and 103 are such as regular user-end computer devices. A program process performed in the terminal device initiates a user interface for assisting the user to join the blockchain network 15 provided by the system. After that, the user can query the records via the user interface and share the records in accordance with the level of authorization according to the blockchain ID of user. The medical staff or care personnel may have the authorization to access the records of patients over the blockchain network and retrieve the records after obtaining a trust in accordance with his blockchain IDs, and then to create the EMR/EHR record after diagnosis and treatment.

In the method for sharing the electronic EMR/EHR records, the following process allows a regular user to look up the medical-health records, and the medical staff to create the records.

It should be noted that the blockchain technology is incorporated into the method for accessing the EMR/EHR records not only for assisting the users to acquire their blockchain IDs and corresponding keys (public key, private key), but also for providing service to look up and share the records. The indexes with respect to the records are created and stored over the decentralized nodes of blockchain network 15. Further, the indexes can also be stored to designated host nodes. Each index over the blockchain network 15 records a corresponding database address. For example, the database address indicates an address for each record in the database, or to an address for a file in an archive. The index acts as a virtual link that is provided as a link to the actual record/file stored in the database at a medical-health institution system. Therefore, the blockchain-based index has advantages of being secure, tamper-proof, and convenient when making queries since the user would need to be firstly verified by the server system 12 using his blockchain ID, obtain the authorization to query the indexes of records, and then retrieve the actual data in the database 14.

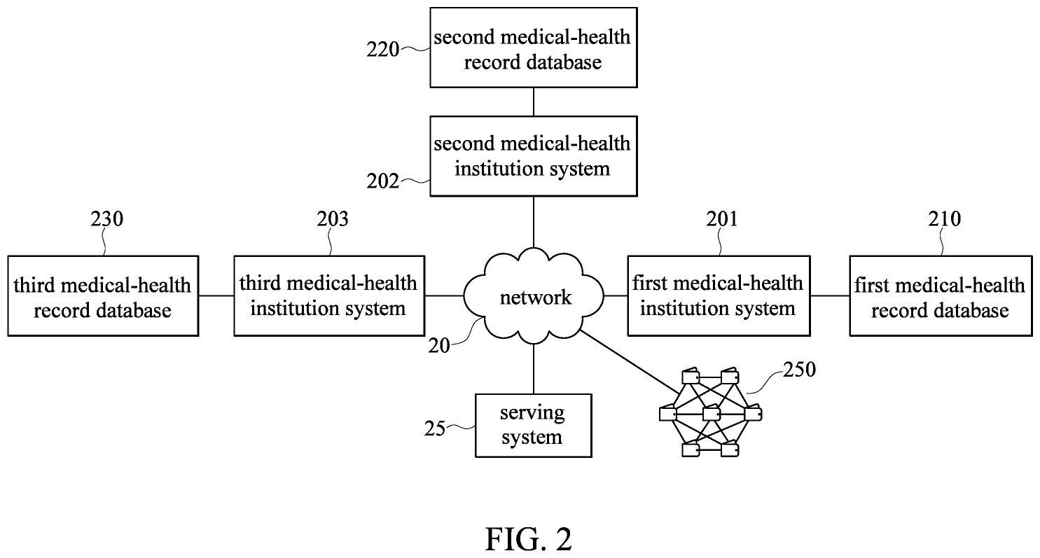

Reference is made to FIG. 2 that shows another system framework for managing the electronic EMR/EHR records using a blockchain technology in one embodiment of the disclosure.

A server system 25 forms a cloud system that connects with one or more medical-health institution systems (201, 202, and 203). The server system 25 links to each of the medical-health record databases (210, 220, 230) of each of the medical-health institution systems (201, 202, 203), or is constituted by the medical-health record databases (210, 220, 230) of the medical-health institution systems (201, 202, 203).

In the present embodiment, the server system 25 links with a plurality of medical-health institution systems (201, 202, and 203). The server system 25 implements a cloud platform and establishes a cloud database that is composed of the medical-health record databases (210, 220, 230) provided by the medical-health institutions. The plurality of medical-health institution systems (201, 202, and 203) form the nodes in a blockchain network. In an exemplary example, the server system 25 connects to a first medical-health institution system 201, a second medical-health institution system 202 and a third medical-health institution system 203 via a network. The system initiates a blockchain 250 for sharing the indexes for the electronic EMR/EHR records. These medical-health institution systems (201, 202, and 203) form the nodes of blockchain 250. The systems (201, 202, and 203) also have their proprietary medical-health record databases that may be represented by the first medical-health record database 210, the second medical-health record database 220 and the third medical-health record database 230.

The system adopts a blockchain technology to record indexes for electronic EMR/EHR records. The medical-health institutions share the indexes for EMR/EHR records in the blockchain 250. Each of the indexes for EMR/EHR records links with a corresponding EMR/EHR record stored in respective ones of the medical-health record databases (210, 220, and 230). An end user such as a patient or a medical staff is able to access the records by the blockchain 250 via a network 20. The system for acquiring the electronic EMR/EHR record integrates various EMR/EHR records from different locations. The system allows every patient to access his EMR/EHR records created at different medical-health institutions. Further, the medical staff can obtain complete EMR/EHR records rather than only part of the records or the records from a single source as may be the case with the conventional technology.

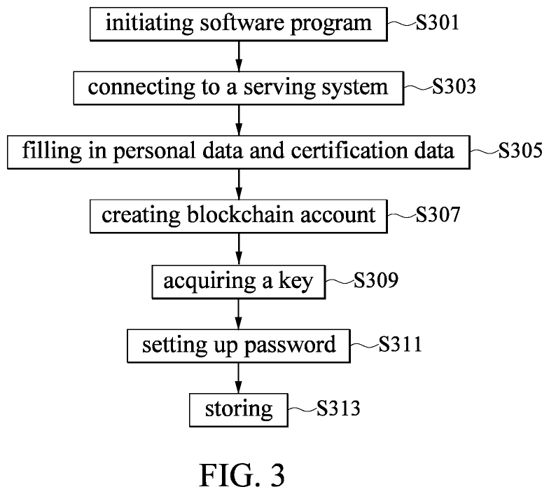

FIG. 3 shows a flow chart illustrating the process for a user to register to a system service. In step S301, the user manipulates a terminal device to perform a software program provided by a system, or a specific program. The terminal device is then connected with a server system (step S303). The server system submits a request for activating a user device by the software program. In step S305, a user can fill in his personal data and verification data using the software program.

In step S307, the system assists the user to create his blockchain account and ID according to his personal data. In step S309, the system also generates a pair of keys including a private key and a public key that allow the user to decrypt and encrypt the blockchain-based data. In step S311, the system may request the user to set up a password for the private key via a user interface. It should be noted that the aspect of the password can be replaced by other aspects of authentication, for example, a fingerprint verification or facial recognition. After the user registers to the system service, the requisite data such as the key is stored into the terminal device (step S313).

In the process of creating the blockchain ID, the system requires the user to install a corresponding software program and provide the verification data. In the meantime, the user can choose a device to participate in the verification process. Further, the software program is configured to be bound with the device for strengthening the security. The device binding can prevent the keys or the identity data from being stolen. According to the settings made during this registration process, a regular user, e.g. the patient, only has permission to access his own EMR/EHR records.

Still further, the medical staff also needs to obtain his own blockchain ID for accessing the EMR/EHR records in the blockchain. In the method for sharing the EMR/EHR records, the medical staff can access the EMR/EHR records of the patient when establishing a trust relationship with the patient. A software program is installed in the device used by the medical staff. When the medical staff is authorized by the patient, the medical staff is able to access the EMR/EHR records in respect of the patient. In an exemplary example, the medical staff can obtain the patient's authorization by scanning an authorization code, e.g. QR Code, provided by the patient. After that, the medical staff can access the records after a verification process according to the patient's authorization and his blockchain ID. Specifically, the medical staff obtains indexes for electronic EMR/EHR records and then accesses the content in the records.

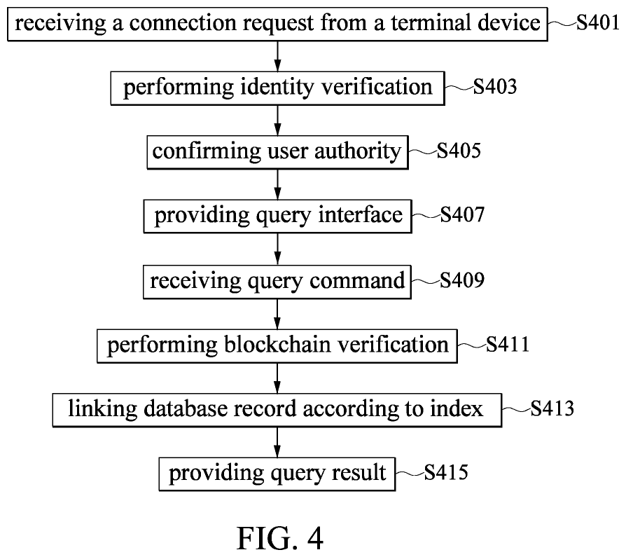

FIG. 4 shows a flow chart describing the method for accessing the EMR/EHR records in one embodiment of the disclosure.

In an initial step S401, a server system receives a connection request generated by a terminal device via a user interface. The connection request includes verification data in connection with a user that may correspond to the user blockchain identification data. Therefore, the server system performs an identity verification process (step S403), and verifies the user authorization according to the user blockchain ID (step S405).

The system then initiates a query interface for the user to query the records through a software procedure (step S407). The user inputs a query criterion such as a date range for obtaining the records within the date range. The records are such as diagnosis data, EMR/EHR data, and/or examination reports. If the user adds query criterions such as a specific medical institution, a doctor name or a specific department, a query command is generated and transmitted to the server system (step S409).

The server system requires the blockchain nodes to verify the records corresponding to the query command based on the user blockchain ID and the query command (step S411). The server system can therefore obtain one or more indexes for the electronic EMR/EHR records. The indexes allow the server system to query the records in a database and obtain the actual data of the records (step S413). The result is then transmitted to the terminal device via a network (step S415). The indexes are tamper-proof since the result is verified by a plurality of blockchain nodes in the process for acquiring the indexes. In the terminal device, a key, e.g. a private key, is required to decrypt the query result so as to obtain the actual data of the records.

In one of the embodiments, when the server system obtains the indexes for the records, the system can link to the actual data of records in the database according to the indexes. The actual data are such as the electronic medical records, health records and/or related files. The one or more indexes form a query result in the system. The query result is provided to the terminal device. Using the one or more indexes, the terminal device can obtain the actual data of the records.

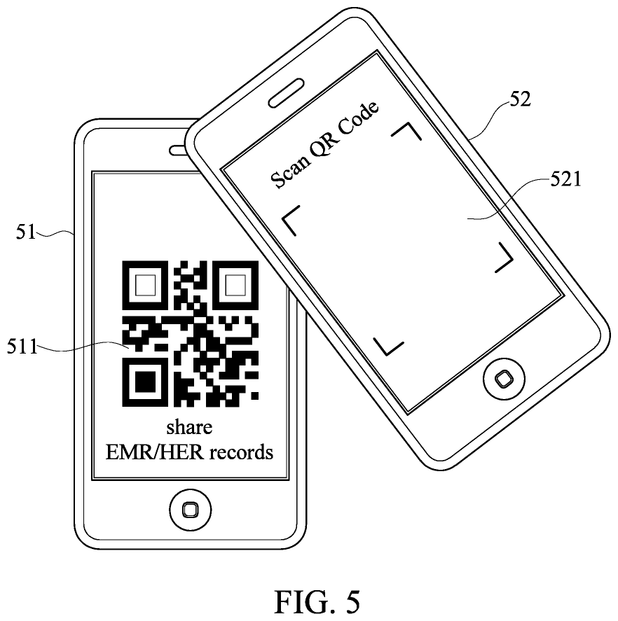

FIG. 5 shows a circumstance where the EMR/EHR records are shared between two mobile devices in one embodiment of the disclosure.

In an exemplary example, when a user, e.g. a patient or his relative, comes before another person such as a doctor or any medical staff, the user may need to share his EMR/EHR records with the person. Other than using a conventional database to share the records, the method for sharing EMR/EHR records according to the present disclosure provides a novel scheme to share the EMR/EHR records. In the method, the user uses a mobile device 51 to acquire the EMR/EHR record indexes when he passes blockchain verification according to a process recited in FIG. 4. The user can select one or more EMR/EHR records to be shared. One or more EMR/EHR records are selected by the user via a user interface. An instruction for sharing the EMR/EHR records is accordingly generated. The serving system then receives the instruction from the terminal device, and a signal for sharing EMR/EHR records that are selected from the one or more EMR/EHR record indexes is generated. The serving system then generates a trust anchor after a decoding process.

The above-mentioned trust anchor is as a two-dimensional bar code 511 displayed on the mobile device 51 in the present example. The two-dimensional bar code 511 is such as a QR code that constitutes a verification data for trust relationship between two persons. In one embodiment, a scanning program 521 is initiated in a mobile device 52 of the medical staff. The two-dimensional bar code 511 is scanned/captured by the scanning program 521 executed in the mobile device 521 of the medical staff. The bar code 511 is decoded for obtaining a trust anchor. The trust anchor is encoded to form an authorization message by a software program. The authorization message is transferred to the serving system and is used to establish a trust relationship to acquire the EMR/EHR records.

The present example uses the two-dimensional bar code 511 to convey the trust anchor to establish the trust relationship. However, the trust relationship may not be limited to using the QR code but a set of trust codes combining English letters and numbers or a numerical node shown in a simple message. Further, the serving system generates the trust anchor with an effective time. In practice, a timestamp is added to the trust anchor for setting up an effective time for the trust relationship, for example 10 minutes. Therefore, the trust anchor becomes invalid when the serving system does not receive the authorization message from the mobile device 52 of the medical staff after the effective time.

In one further embodiment, if the trust relationship is established by scanning the two-dimensional bar code 511, the system can require the mobile device 51 of the user and the mobile device 52 of the medical staff to be located within a certain range. The serving system can obtain the locations of the mobiles devices 51 and 52 through the positioning technologies such as GPS positioning and indoor positioning. Therefore, a trust location data is defined. When the serving system determines a distance between the terminal device, e.g. the mobile device 51, and shared person, e.g. the mobile device 52, is beyond a certain range, the trust anchor becomes invalid. Thus, a security threshold is therefore provided.

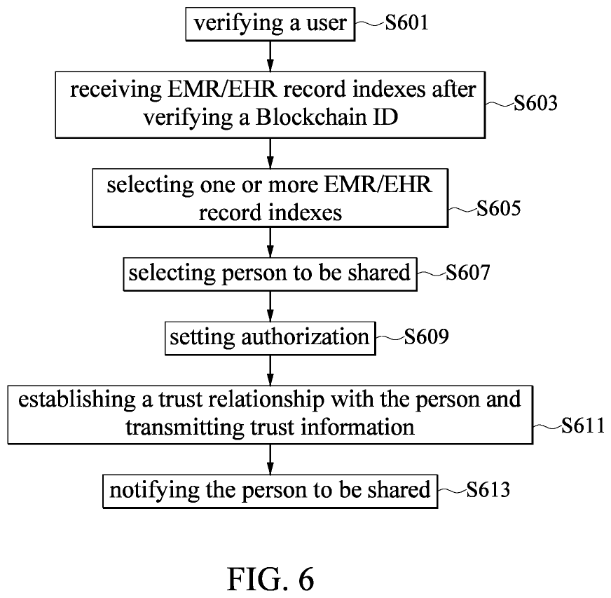

FIG. 6 shows a flow chart describing a method for sharing EMR/EHR records in one embodiment of the disclosure.

In the process, a user first manipulates a terminal device, e.g. the mobile device 51 shown in FIG. 5, to perform verification. A connection request for connecting with a serving system is generated. The serving system verifies the data sent by the terminal device, and also verifies the user blockchain ID by blockchain technology (step S601).

Through the serving system, the terminal device acquires the EMR/EHR record indexes recorded in the blockchain nodes after a blockchain verification process (step S603). In the meantime, the one or more EMR/EHR record indexes in accordance with the blockchain ID can be shown on the user's terminal device. The EMR/EHR record indexes can be presented in a time sequence. The user then selects one or more EMR/EHR record indexes to be shared. The selected one or more EMR/EHR records can be records occurring within a time interval (step S605). A list showing other blockchain users, e.g. the medical staffs, is provided for the user to select one or more shared persons (step S607). Alternatively, the user can fill in the name of the shared person into a field. Further, the user can also exchange the information with the shared person. If one or more shared persons are selected, an authorization condition can be set (step S609). In addition to the blockchain ID of each of the shared persons, the authorization condition can include an effective time for sharing the records. The authorization condition will fail if the effective time expires.

After that, the terminal device generates an instruction for sharing the EMR/EHR records according to the above-described conditions, and a signal for sharing the EMR/EHR records that are selected from the one or more EMR/EHR record indexes. The instruction and signal are then transmitted to the serving system. When the serving system receives the instruction and the signal for sharing the EMR/EHR records, the serving system generates a trust anchor and applies an effective time to the trust anchor. In an example, the trust anchor being transmitted to the terminal device can be encoded as a two-dimensional bar code.

The trust anchor is then transmitted to the terminal device. A shared person can use his device to obtain the trust anchor by a scanning program so as to establish a trust relationship between the user and the shared person (step S611). The device of the shared person encodes the trust anchor as an authorization message that is then transmitted to the serving system. The serving system can verify a blockchain ID of the shared person and the authorization message received from the device of the shared person. After that, a notification is generated and sent to the shared person (step S613). The related EMR/EHR records are also transmitted to the device of the shared person. It should be noted that the authorization message is the message sent from the device of the shared person to the serving system, and the message is generated by encrypting the trust anchor and the blockchain ID of the shared person.

Further, in addition to using the two-dimensional bar code shown in FIG. 5 to establish the trust relationship between two parties, another way is to set up a list of trusted persons in the serving system for sharing the EMR/EHR records. For example, in one embodiment of the disclosure, the signal for sharing the EMR/EHR records that is configured to be transmitted from the terminal device to the serving system includes blockchain ID for one or more shared persons. Therefore, the serving system can directly set up the one or more shared persons. The EMR/EHR record indexes are transmitted to one or more shared persons afterwards.

After that, the shared person can acquire the one or more EMR/EHR record indexes according to the blockchain addresses that are configured to be shared. In the meantime, the medical staff can obtain the EMR/EHR records using his private key due to the trust relationship.

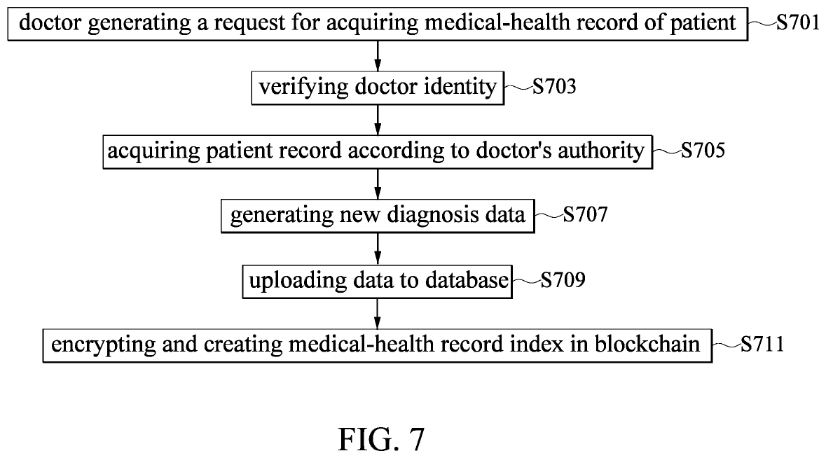

According to the above descriptions regarding the method for sharing the EMR/EHR records, the medical-health record indexes are continuously created over the blockchain based on a time sequence. Relevant procedures can be referred to in FIG. 7 that shows a flow chart describing a process operated in a device of the medical staff for establishing the EMR/EHR records in one embodiment of the disclosure.

In step S701 of the process, a doctor-end device generates a request for acquiring a medical-health record of a patient. For example, when a medical staff, e.g., the doctor, inquires the patient about his condition, the medical staff may need to obtain the patient's EMR/EHR records. After obtaining the patient's authorization, the medical staff operates the device to generate a request for accessing the EMR/EHR records. When the server system receives the request, in step S703, the server system firstly verifies the medical staff identity. The server system can verify the blockchain ID of the medical staff and the records in the request according to the verification data sent by the medical staff. The verification data can include a blockchain ID, the patient identity, and authorization data. Next, the server system acquires the EMR/EHR records in accordance with the authorization related to the request (step S705).

It is possible to generate new diagnosis data, medical data, and/or examination reports when the medical staff conducts the inquiry and diagnosis (step S707). The newly-added electronic EMR/EHR records can also be encrypted using a public key of the patient and uploaded to the database (step S709). In step S711, the indexes for indicating the medical-health records in the database are created. The indexes are then encrypted to form the records distributed over the blockchain network.

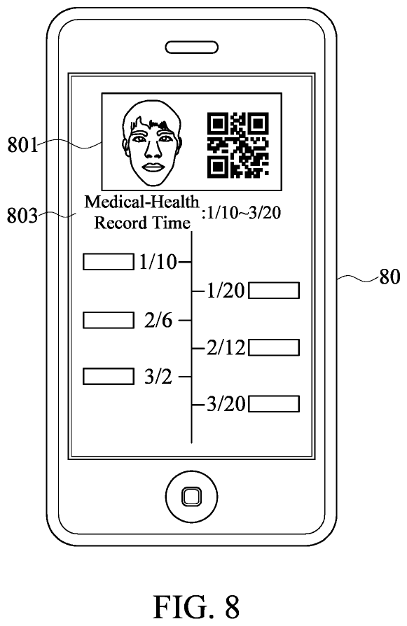

Reference is made to FIG. 8, which shows a schematic diagram depicting a screenshot showing the electronic EMR/EHR records obtained from the database. A screenshot similar to that shown in FIG. 8 can also be shown on another device when the records are shared with this device via the indexes.

In the diagram, a personal blockchain data 801 and the EMR/EHR records (803) within a time period obtained by the terminal device 80 after acquiring the indexes are shown. The shown records also include information such as a patient ID, timestamps, and other contents in the records. The terminal device 80 displays the one or more records within a time period via a user interface. In an exemplary example, the records shown on an APP screen of the device include recent record indexes or record indexes within a time period designated by the user that are arranged on the screen in a chronological order.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for sharing EMR/EHR records, operated in a serving system having a database, comprising:
receiving a connection request from a terminal device;
verifying a blockchain ID of a user of the terminal device;
providing one or more EMR/EHR record indexes in accordance with the blockchain ID;
receiving an instruction for sharing EMR/EHR records from the terminal device, and generating a signal for sharing EMR/EHR records that are selected from the one or more EMR/EHR record indexes;
generating a trust anchor and transmitting the trust anchor to the terminal device;
receiving an authorization message from a device of a shared recipient to whom the one or more EMR/EHR records are shared, wherein the device of the shared recipient obtains the trust anchor from the terminal device so as to form the authorization message;
verifying an blockchain ID of the shared recipient with the one or more EMR/EHR records and the authorization message; and
transmitting the EMR/EHR records to the device of the shared recipient with the one or more EMR/EHR records after the verification is done.

2. The method according to claim 1, wherein the trust anchor is encoded as a two-dimensional bar code, and the two-dimensional bar code is provided for the terminal device and allowing the device of the shared recipient with the one or more EMR/EHR records to acquire the trust anchor by a scanning program.

3. The method according to claim 2, wherein the serving system receives the blockchain ID of the user and the shared recipient with the one or more EMR/EHR records via a user interface initiated by a software program executed in the terminal device so as to verify the blockchain ID and provide the one or more EMR/EHR record indexes according to the blockchain ID.

4. The method according to claim 3, wherein the serving system forms a cloud system that connects one or more medical-health institution systems, the database of the serving system connects with every medical-health record database of each of the medical-health institution systems, or the database of the serving system is composed of a plurality of medical-health record databases of the medical-health institution systems; a database address is recorded by the EMR/EHR record index distributed over a blockchain network, and the database address is used to link to an actual EMR/EHR record or file in the medical-health record database of each of the medical-health institution systems.

5. The method according to claim 1, wherein the signal for sharing EMR/EHR records includes one or more EMR/EHR records and an effective time for sharing.

6. The method according to claim 5, wherein the signal for sharing EMR/EHR records further includes the blockchain ID of the shared recipient with the one or more EMR/EHR records.

7. The method according to claim 6, wherein the trust anchor is encoded as a two-dimensional bar code, and the two-dimensional bar code is provided for the terminal device and allowing the device of the shared recipient with the one or more EMR/EHR records to acquire the trust anchor by a scanning program.

8. The method according to claim 7, wherein the serving system receives the blockchain ID of the user and the shared recipient with the one or more EMR/EHR records via a user interface initiated by a software program executed in the terminal device so as to verify the blockchain ID and provide the one or more EMR/EHR record indexes according to the blockchain ID.

9. The method according to claim 8, wherein the serving system forms a cloud system that connects one or more medical-health institution systems, the database of the serving system connects with every medical-health record database of each of the medical-health institution systems, or the database of the serving system is composed of a plurality of medical-health record databases of the medical-health institution systems; a database address is recorded by the EMR/EHR record index distributed over a blockchain network, and the database address is used to link to an actual EMR/EHR record or file in the medical-health record database of each of the medical-health institution systems.

10. The method according to claim 1, wherein the authorization message transmitted by the device of the shared recipient with the one or more EMR/EHR records is a message sent from the device of the shared recipient with the one or more EMR/EHR records to the serving system, and the message is generated by encrypting the trust anchor and the blockchain ID of the person.

11. The method according to claim 10, wherein the trust anchor has an effective time, and the trust anchor is invalid when the serving system does not receive the authorization message upon expiry of the effective time.

12. The method according to claim 11, wherein the trust anchor has a trust location data, and the trust anchor is invalid when the serving system determines that a distance between the terminal device and the device of the shared recipient with the one or more EMR/EHR records is beyond a range.

13. The method according to claim 12, wherein the trust anchor is encoded as a two-dimensional bar code, and the two-dimensional bar code is provided for the terminal device and allowing the device of the person to acquire the trust anchor by a scanning program.

14. The method according to claim 13, wherein the serving system receives the blockchain ID of the user and the shared recipient with the one or more EMR/EHR records via a user interface initiated by a software program executed in the terminal device so as to verify the blockchain ID and provide the one or more EMR/EHR record indexes according to the blockchain ID.

15. The method according to claim 14, wherein the serving system forms a cloud system that connects one or more medical-health institution systems, the database of the serving system connects with every medical-health record database of each of the medical-health institution systems, or the database of the serving system is composed of a plurality of medical-health record databases of the medical-health institution systems; a database address is recorded by the EMR/EHR record index distributed over a blockchain network, and the database address is used to link to an actual EMR/EHR record or file in the medical-health record database of each of the medical-health institution systems.

16. A system for sharing EMR/EHR records, comprising:
a serving system having a database; and
a software program stored in a terminal device, used for connecting with the serving system for obtaining one or more EMR/EHR record indexes when the software program is executed by a processor of the terminal device, and generating an instruction for sharing EMR/EHR records and a signal for sharing EMR/EHR records;
wherein the serving system operating a method for sharing EMR/EHR records comprising:
receiving a connection request from the terminal device;
verifying a blockchain ID of a user of the terminal device;
providing the one or more EMR/EHR record indexes in accordance with the blockchain ID;
receiving an instruction for sharing EMR/EHR records from the terminal device, and generating a signal for sharing EMR/EHR records that are selected from the one or more EMR/EHR record indexes;
generating a trust anchor and transmitting the trust anchor to the terminal device;
receiving an authorization message from a device of a shared recipient with the one or more EMR/EHR records, wherein the device of the shared recipient obtains the trust anchor from the terminal device so as to form the authorization message;
verifying a blockchain ID of the shared recipient with the one or more EMR/EHR records and the authorization message; and
transmitting the EMR/EHR records to the device of the shared recipient with the one or more EMR/EHR records after the verification is done.

17. The system according to claim 16, wherein the signal for sharing EMR/EHR records includes one or more EMR/EHR records, an effective time for sharing, and the blockchain ID of the shared recipient with the one or more EMR/EHR records.

18. The system according to claim 17, wherein the trust anchor is encoded as a two-dimensional bar code, and the two-dimensional bar code is provided for the terminal device and allowing the device of the shared recipient with the one or more EMR/EHR records to acquire the trust anchor by a scanning program.

19. The system according to claim 18, wherein the serving system receives the blockchain ID of the user and the shared recipient with the one or more EMR/EHR records via a user interface initiated by a software program executed in the terminal device so as to verify the blockchain ID and provide the one or more EMR/EHR record indexes according to the blockchain ID.

20. The system according claim 19, wherein the serving system forms a cloud system that connects one or more medical-health institution systems, the database of the serving system connects with every medical-health record database of each of the medical-health institution systems, or the database of the serving system is composed of a plurality of medical-health record databases of the medical-health institution systems; a database address is recorded by the EMR/EHR record index distributed over a blockchain network, and the database address is used to link to an actual EMR/EHR record or file in the medical-health record database of each of the medical-health institution systems.
